(19) United States
(12) Patent Application Publication
(10) Pub. No.: US 2015/0064281 A1
(43) Pub. Date: Mar. 5, 2015

(54) OPHTHALMIC COMPOSITION

(71) Applicant: ROHTO PHARMACEUTICAL CO., LTD., Osaka -shi, Osaka (JP)

(72) Inventors: Kazuhiro Fukushima, Osaka-shi (JP); Mariyo Kato, Osaka-shi (JP); Yoshihiro Takai, Osaka-shi (JP); Masashi Itoh, Osaka-shi (JP)

(73) Assignee: ROHTO PHARMACEUTICAL CO., LTD., Osaka-shi, Osaka (JP)

(21) Appl. No.: 14/391,559

(22) PCT Filed: Apr. 4, 2013
(86) PCT No.: PCT/JP2013/060288

§ 371 Date: Oct. 9, 2014

(30) Foreign Application Priority Data
Apr. 10, 2012 (JP) 2012-089541

(51) Int. Cl. A61K 31/155; A61K 33/30

(52) U.S. Cl. CPC A61K 31/155; A61K 33/30
USPC 424/641

(57) ABSTRACT

The present invention provides an ophthalmic composition containing (A) a biguanide-based disinfectant and (B) zinc chloride. According to the present invention, an ophthalmic composition having remarkably increased disinfection effects against Acanthamoeba is provided.

### TECHNICAL FIELD

The present invention relates to an ophthalmic composition which can exhibit high disinfectant effects against Acanthamoeba. In addition, the present invention relates to an ophthalmic composition having high safety in the aspects of improving corneal barrier functions and preventing invasion of a pathogen such as Acanthamoeba. Further, the present invention relates to an ophthalmic composition which is less likely to cause size changes of contact lenses even when the lenses are immersed for a long period of time, so that the ophthalmic composition can be used safely against contact lenses. In addition, the present invention relates to an anti Acanthamoeba agent, a method for disinfecting against Acanthamoeba, a method for improving corneal barrier functions, a method for inhibiting size changes of contact lenses caused by boric acid and/or a salt thereof, and the like.

### BACKGROUND ART

Acanthamoeba corneal infectious diseases (Amoeba keratitis) are corneal infectious diseases which are onset by infecting the cornea with Acanthamoeba, for example, Acanthamoeba castellanii, Acanthamoeba polyphaga or the like, which is an intractable corneal disease that leaves a strong opacity in the cornea. Acanthamoeba is a kind of protistas existing widely in soils, freshwater, or the like, which cannot easily deposit on a healthy cornea, but if the cornea is damaged by the use of contact lenses or the like, it is considered that the protistas deposit thereto and cause infections. Moreover, in fact, many of the patients suffering from Acanthamoeba corneal infectious diseases are those individuals with experiences in wearing contact lenses, and it is said that use of the Acanthamoeba-deposited contact lenses is a main infection causation.

To date, a magical agent for the treatment against Acanthamoeba corneal infectious diseases has not been known. Therefore, an antifungal agent has been presently used in the treatment of Acanthamoeba corneal infectious diseases, but there are some cases where sufficient therapeutic effects cannot be obtained even when an antifungal agent is used, so that the treatment of scraping off the infected corneal surface is further necessitated. Also, the eradication of the Acanthamoeba corneal infectious diseases would require a long period of time of several months, and when not eradicated, in some cases corneal transplantation is inevitably performed. As such, it is difficult to eradicate the disease once an individual suffers from an Acanthamoeba corneal infectious disease, so that the prevention of the Acanthamoeba corneal infectious diseases is considered to be important.

Conventionally, it has been tried to give Acanthamoeba-disinfectant action to an ophthalmic composition such as a solution for contact lenses care. For example, it is reported in Patent Publication 1 that a solution composition for contact lenses containing a proteolytic enzyme, an anionic surfactant, a non-reducing polyhydric alcohol, a borate-based buffer and a water-soluble polymer compound each in a given amount exhibits disinfectant effects against Acanthamoeba. In addition, it is reported in Patent Publication 2 that a polylysine is effective in disinfection against Acanthamoeba for contact lenses other than high-water-content, ionic water-containing soft contact lenses (group IV lenses).

Further, Patent Publication 3 describes that a cationic antiseptic such polyhexamethylene biguanide, which is also referred to as “PHMB,” is used for inhibiting infections against Acanthamoeba though to a limited effect. PHMB is considered to have a relatively high safety against eyes, as seen in its use as a disinfectant in swimming pools in the USA, or the like. In the recent years, it is pointed out that there is a possibility that polyhexamethylene biguanide in a combination with a specified contact lens causes an ectocorneal disorder which is called corneal staining. Therefore, there are some dilemmas in that blending

PHMB in a high concentration in an ophthalmic composition in order to exhibit a high anti-Acanthamoeba action might increase an onset risk of an ectocorneal disorder, which in turn rather makes the cornea into a state that is more easily infected by Acanthamoeba.

On the other hand, a method for inhibiting proliferation of Acanthamoeba using a polyvalent cationic chelating agent that chelates polyvalent cations (zinc or the like) which function as a co-factor of an enzyme necessary for the metabolism in protozoans has been known (Patent Publication 4).

### PRIOR ART REFERENCES

### Patent Publications

Patent Publication 1: Japanese Patent Laid-Open No. 2003-57610

Patent Publication 2: Japanese Patent Laid-Open No. 2002-143277

Patent Publication 3: WO 02/49633

Patent Publication 4: Japanese Patent Laid-Open No. Hei-9-506518

### SUMMARY OF THE INVENTION

### Problems to be Solved by the Invention

In view of the importance in the prevention of Acanthamoeba corneal infectious diseases, the development of a more useful ophthalmic composition having high disinfectant effects against Acanthamoeba has been desired. In view of the conventional problems, a main object of the present invention is to provide a useful ophthalmic composition having remarkable disinfectant effects against Acanthamoeba.

### Means to Solve the Problems

The present inventor has made intensive studies in order to accomplish the objects mentioned above. As a result, according to an ophthalmic composition containing a biguanide-based disinfectant together with zinc chloride, the present inventors have found that remarkably high disinfectant effects against Acanthamoeba are obtained. In addition, as a result of further studies, the present inventors have also found that by a combined use of a biguanide-based disinfectant and zinc chloride, the corneal barrier functions can be effectively improved, so that invasions of Acanthamoeba can be prevented, so that such a composition can be used as a very safe ophthalmic composition even when the composition is directly applied to the eyes. On the other hand, in a case where contact lenses are subjected to an immersion treatment in an ophthalmic composition containing boric acid and/or a salt thereof for a long period of time, there are some disadvantages that lenses are more likely to be expanded. In view of the above, according to the present invention, it has been found that a combined use of a biguanide-based disinfectant and zinc chloride in an ophthalmic composition containing boric acid and/or a salt thereof at a given concentration allows to inhibit size changes caused by immersing contact lenses in an ophthalmic composition containing boric acid and/or a salt thereof over a long period of time, so that the contact lenses can be safely used. The present invention has been perfected as a result of further improvements based on these findings.

In other words, the present invention provides an ophthalmic composition of the embodiments listed hereinbelow.
Item 1-1: An ophthalmic composition characterized in that the ophthalmic composition contains (A) a biguanide-based disinfectant, and (B) zinc chloride.
Item 1-2: The ophthalmic composition according to the item 1-1, wherein the biguanide-based disinfectant (A) is a compound having at least one biguanide group represented by the formula: —NH—C(═NH)—NH—C(═NH)—NH—.
Item 1-3. The ophthalmic composition according to the item 1-1 or 1-2, wherein the biguanide-based disinfectant (A) contains at least one member selected from the group consisting of hexamethylene biguanide, a polymer thereof, chlorhexidine, alexidine, and salts thereof.
Item 1-4. The ophthalmic composition according to any one of claims 1-1 to 1-3, wherein the component (A) is contained in a total content of from 0.000001 to 0.01 w/v %.
Item 1-5. The ophthalmic composition according to any one of claims 1-1 to 1-4, wherein the component (B) is contained in a total content of from 0.0001 to 0.05 w/v %.
Item 1-6. The ophthalmic composition according to any one of claims 1-1 to 1-5, wherein a total content of the component (B) is from 10 to 50,000 parts by weight, based on 100 parts by weight of a total content of the component (A).
Item 1-7. The ophthalmic composition according to any one of the items 1-1 to 1-6, further containing (C) at least one member selected from the group consisting of boric acid and salts thereof.
Item 1-8. The ophthalmic composition according to any one of the items 1-1 to 1-7, wherein the component (C) is contained, calculated as a boron atom content, in a proportion of from 0.0001 to 0.045 mol/100 mL.
Item 1-9. The ophthalmic composition according to any one of the items 1-1 to 1-8, further containing a nonionic surfactant.
Item 1-10. The ophthalmic composition according to the item 1-9, wherein the nonionic surfactant is a polyoxyethylene sorbitan fatty acid ester, a polyoxyethylene hydrogenated castor oil, or a polyoxyethylene-polyoxypropylene block copolymer.
Item 1-11. The ophthalmic composition according to any one of claims 1-1 to 1-10, for use in disinfection against Acanthamoeba.
Item 1-12. The ophthalmic composition according to any one of claims 1-1 to 1-11, wherein the ophthalmic composition is in a pharmaceutical dosage form that is directly applicable to a corneal surface.
Item 1-13. The ophthalmic composition according to any one of claims 1-1 to 1-12, wherein the ophthalmic composition is an ophthalmic composition for contact lenses.
Item 1-14. The ophthalmic composition according to the item 1-13, wherein the contact lenses are soft contact lenses.
Item 1-15. The ophthalmic composition according to the item 1-13 or 1-14, wherein the contact lenses are soft contact lenses in which soft lenses classification is Group IV, or silicone hydrogel contact lenses.
Item 1-16. The ophthalmic composition according to any one of claims 1-13 to 1-15, wherein the ophthalmic composition for contact lenses is a contact lens care agent.
Item 1-17. The ophthalmic composition according to any one of claims 1-13 to 1-16, wherein the ophthalmic composition for contact lenses are a disinfection-washing-storage solution for contact lenses.

In addition, the present invention provides an anti-Acanthamoeba agent of the embodiment listed hereinbelow.
Item 2. An anti-Acanthamoeba agent containing (A) a biguanide-based disinfectant, and (B) zinc chloride.

Also, the present invention provides a method for disinfecting against Acanthamoeba, a method for enhancing a disinfectant action against Acanthamoeba, and a method for giving an ophthalmic composition with a disinfectant action against Acanthamoeba of the embodiments listed hereinbelow.
Item 3. A method for disinfecting against Acanthamoeba, characterized by disinfecting against Acanthamoeba with an ophthalmic composition containing (A) a biguanide-based disinfectant and (B) zinc chloride.
Item 4. A method for enhancing a disinfectant action against Acanthamoeba, characterized by blending (B) zinc chloride in an ophthalmic composition containing (A) a biguanide-based disinfectant.
Item 5. A method for giving an ophthalmic composition with a disinfectant action against Acanthamoeba, characterized by blending (A) a biguanide-based disinfectant and (B) zinc chloride in an ophthalmic composition.

Also, the present invention provides a method for preparing an ophthalmic composition having improved corneal barrier functions, and a method for giving an action of improving corneal barrier functions to an ophthalmic composition of the embodiments listed hereinbelow.
Item 6. A method for preparing an ophthalmic composition having improved corneal barrier functions, characterized by blending (A) a biguanide-based disinfectant and (B) zinc chloride in an ophthalmic composition.
Item 7. A method for giving an action of improving corneal barrier functions to an ophthalmic composition, characterized by blending (B) zinc chloride in an ophthalmic composition containing (A) a biguanide-based disinfectant.

Further, the present invention provides a method for inhibiting size changes of contact lenses caused by boric acid and/or a salt thereof, and a method for giving an action of inhibiting size changes of contact lenses caused by boric acid and/or a salt thereof to an ophthalmic composition of the embodiments listed hereinbelow.
Item 8. A method for inhibiting size changes of contact lenses caused by boric acid and/or a salt thereof, characterized by blending (A) a biguanide-based disinfectant and (B) zinc chloride in an ophthalmic composition containing (C) at least one member selected from the group consisting of boric acid and salts thereof.
Item 9. A method for giving an action of inhibiting size changes of contact lenses caused by boric acid and/or a salt thereof to an ophthalmic composition, characterized by blending (A) a biguanide-based disinfectant and (B) zinc chloride in an ophthalmic composition containing (C) at least one member selected from the group consisting of boric acid and salts thereof.

Further, the present invention also provides the use of the embodiments listed hereinbelow.
Item 10. Use of (A) a biguanide-based disinfectant and (B) zinc chloride in the manufacture of an ophthalmic composition.
Item 11. The use according to the item 10, wherein the ophthalmic composition is a composition as defined in any one of the above items 1-1 to 1-17.

Further, the present invention also provides the use of the embodiments listed hereinbelow.
Item 12. Use of a composition containing (A) a biguanide-based disinfectant and (B) zinc chloride, as an ophthalmic composition.
Item 13. The use according to the item 12, wherein the ophthalmic composition is a composition as defined in any one of the above items 1-1 to 1-17.

Further, the present invention also provides the composition of the embodiments listed hereinbelow.
Item 14. A composition containing (A) a biguanide-based disinfectant and (B) zinc chloride, for use in an ophthalmic composition.
Item 15. The composition according to the item 14, which is as defined in any one of the above items 1-1 to 1-17.

Further, the present invention also provides a method for producing an ophthalmic composition of the embodiments listed hereinbelow.
Item 16. A method for producing an ophthalmic composition, including adding (A) a biguanide-based disinfectant and (B) zinc chloride to a carrier containing water.
Item 17. The method according to the item 16, wherein the ophthalmic composition is a composition as defined in any one of the above items 1-1 to 1-17.

### Effects of the Invention

The ophthalmic composition of the present invention can exhibit remarkably high disinfectant effects against Acanthamoeba. Therefore, according to the ophthalmic composition of the present invention, Acanthamoeba corneal infectious diseases can be prevented at a high level. Further, the ophthalmic composition of the present invention is excellent in the prevention of Acanthamoeba corneal infectious diseases, from the viewpoint that the corneal barrier functions can be effectively improved, so that the ophthalmic composition can be used with high safety. In addition, the ophthalmic composition of the present invention can effectively inhibit size changes of contact lenses, which are likely to be caused when contact lenses are immersed in an ophthalmic composition containing boric acid and/or a salt thereof, so that risks of ectocorneal disorders accompanied by the use of contact lenses can be greatly reduced. The lowering of the corneal barrier functions and the ectocorneal disorders would make the invasions of Acanthamoeba into the cornea easy, which are also causations of increasing risks of infectious diseases. Therefore, the ophthalmic composition of the present invention is effective in the prevention of Acanthamoeba corneal infection diseases at a high level, from the viewpoint that the lowering of the corneal barrier functions and size changes of the contact lenses can be effectively inhibited.

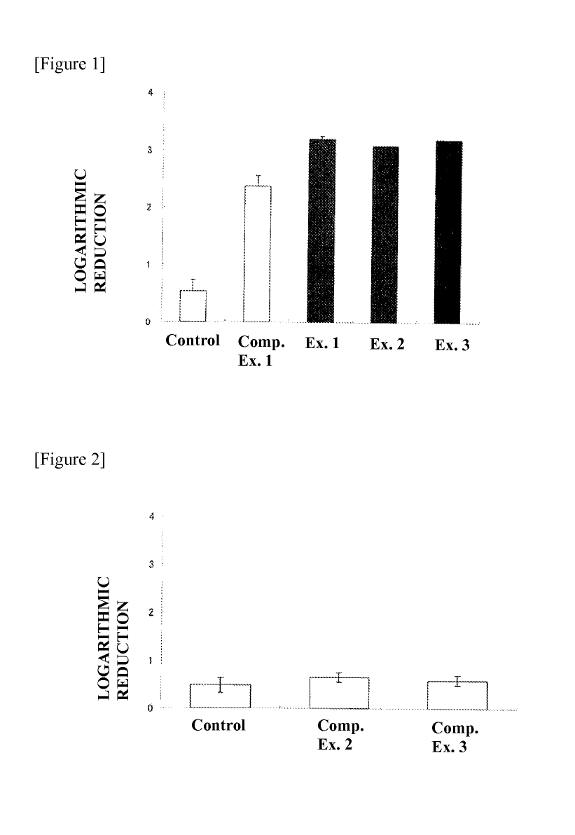

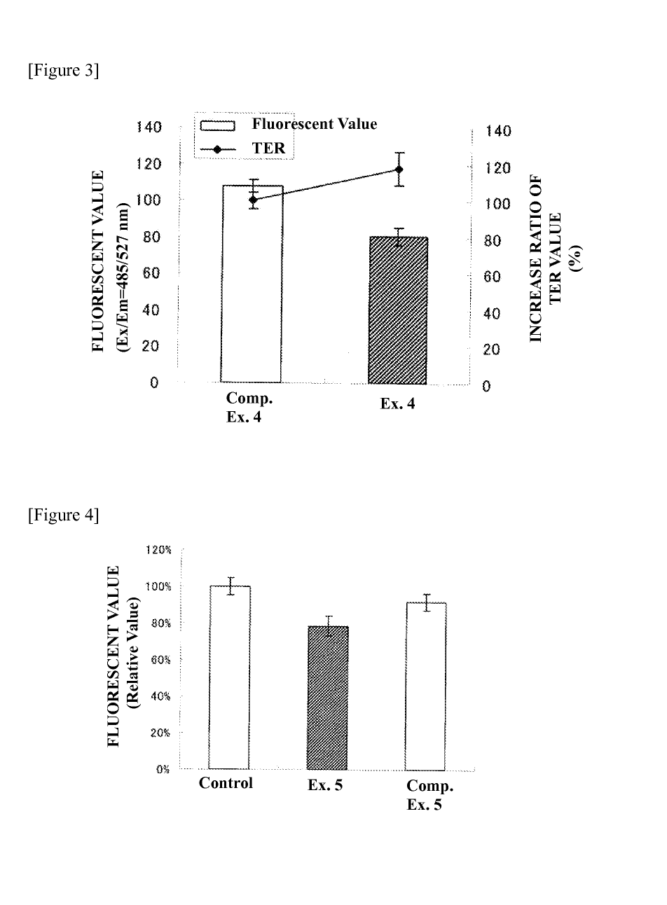

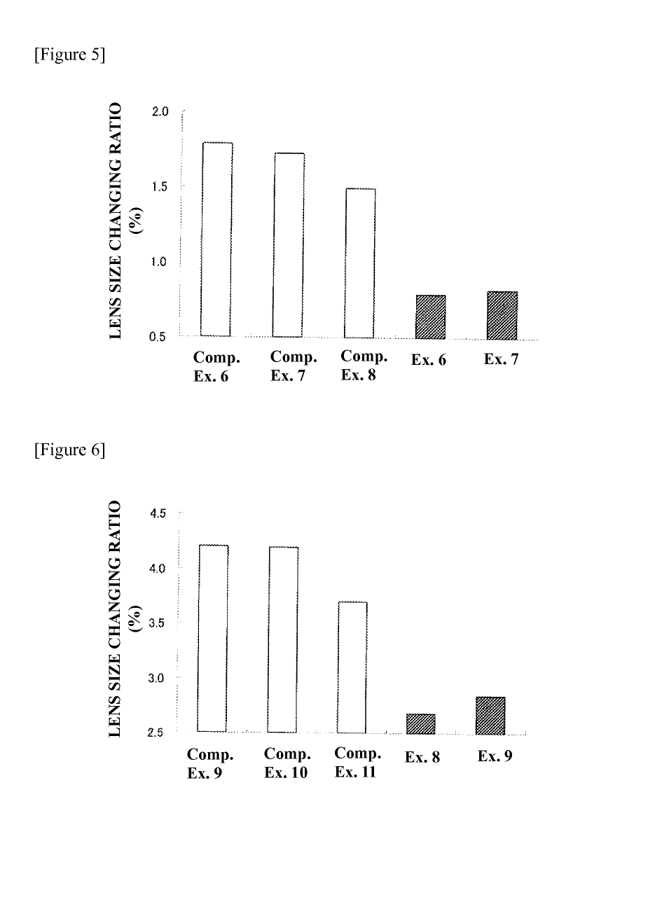

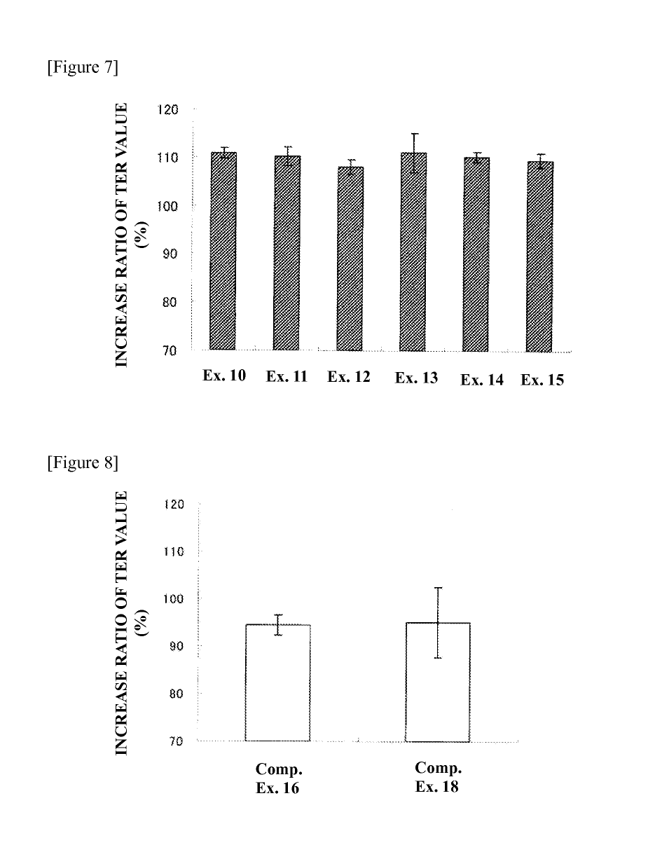

### BRIEF DESCRIPTION OF THE DRAWINGS

[FIG. 1] A graph showing the results of evaluating disinfectant effects against Acanthamoeba of test solutions (Examples 1 to 3 and Comparative Example 1) in Test Example 1.

[FIG. 2] A graph showing the results of evaluating disinfectant effects against Acanthamoeba of test solutions (Comparative Examples 2 and 3) in Referential Test Example 1.

[FIG. 3] A graph showing the results of evaluating influences on barrier functions of ectocorneal cells with respect to test solutions (Example 4 and Comparative Example 4) in Test Example 2.

[FIG. 4] A graph showing the results of evaluating influences on barrier functions of ectocorneal cells with respect to test solutions (Example 5 and Comparative Example 5) in Test Example 3.

[FIG. 5] A graph showing the results of evaluating influences on size changes of contact lenses with respect to test solutions (Examples 6 and 7 and Comparative Examples 6 to 8) in Test Example 4.

[FIG. 6] A graph showing the results of evaluating influences on size changes of contact lenses with respect to test solutions (Examples 8 and 9 and Comparative Examples 9 to 11) in Test Example 5.

[FIG. 7] A graph showing the results of evaluating influences on barrier functions of ectocorneal cells with respect to test solutions (Examples 10 to 15) in Test Example 6.

[FIG. 8] A graph showing the results of evaluating influences on barrier functions of ectocorneal cells with respect to test solutions (Comparative Examples 16 and 18) in Referential Test Example 2.

### MODES FOR CARRYING OUT THE INVENTION

1. Ophthalmic Composition

The ophthalmic composition of the present invention contains a biguanide-based disinfectant, which may be hereinafter expressed as the component (A).

The biguanide-based disinfectant refers to a compound having at least one biguanide group represented by the formula: —NH—C(═NH)—NH—C(═NH)—NH—, which is a known disinfectant in the form of a monomer having at least one biguanide group, the polymer constituted by the monomers, and compounds in the salt form thereof, which may be produced by a known method, or can be obtained as a commercially available product.

The biguanide-based disinfectant usable in the present invention is not particularly limited, so long as the biguanide-based disinfectant is pharmacologically (pharmaceutically) or physiologically acceptable in the field of medicine. For example, the biguanide-based disinfectant is exemplified by the compounds represented by the following general formulas (II), (III), (IV) and (V). Among them, the compounds represented by the general formulas (II) and (III) may be in the form of salts.

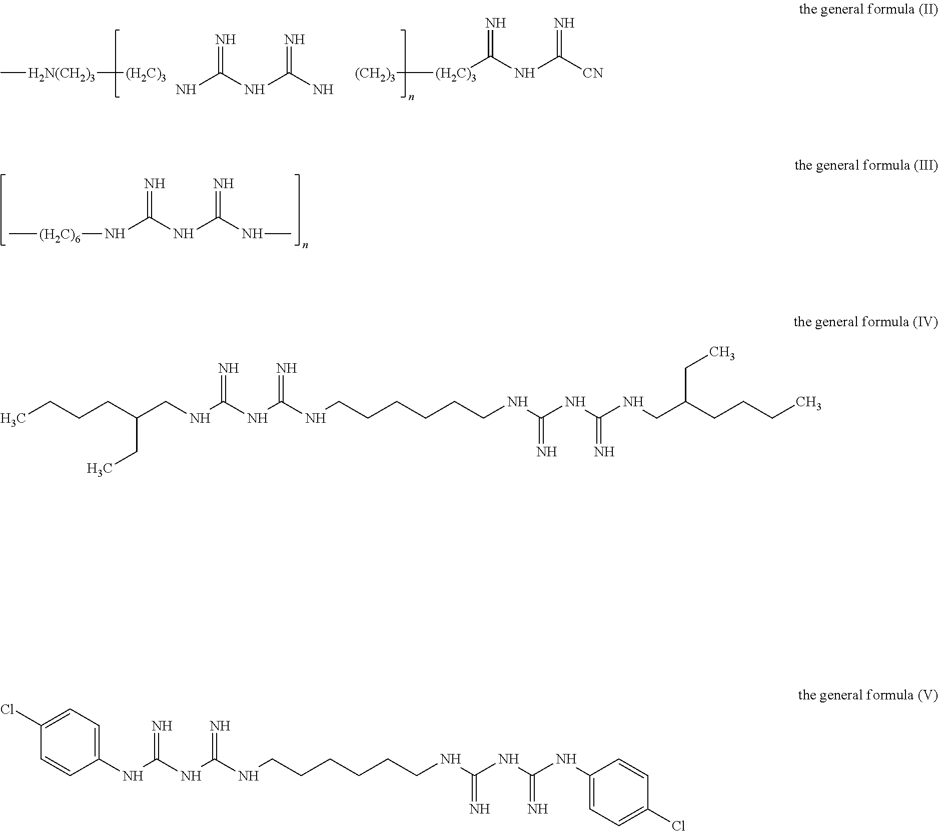

In the general formulas (II) and (III), n is an integer of 1 or more, preferably an integer of from 1 to 500, more preferably an integer of from 1 to 40, and especially preferably from in integer of from 10 to 13.

Concrete examples of the biguanide-based disinfectant usable in the present invention include hexamethylene biguanide and polymers thereof, i.e. polyhexamethylene biguanide (abbreviation: PHMB)), chlorhexidine, alexidine, hexetidine, and the like, and preferably exemplified by hexamethylene biguanide and a polymer thereof, from the viewpoint of even more enhancing a disinfectant action against Acanthamoeba. The hexamethylene biguanide represented by the formula (II) and a polymer thereof are preferred, from the viewpoint of inhibiting the lowering of corneal barrier functions, and inhibiting size changes of contact lenses when boric acid and/or a salt thereof is used.

The biguanide-based disinfectant may be a mixture of a monomer having at least one biguanide group or a polymer thereof. A mixture containing a monomer and polymers having various degrees of polymerization described above, and a mixture containing polymers having various degrees of polymerization described above may also be collectively referred to as polybiguanide. For example, a mixture of a monomer of hexamethylene biguanide and a polymer thereof having various degrees of polymerization may also be collectively referred to as polyhexamethylene biguanide. When the mixture as mentioned above is used, an average degree of polymerization within the numerical range defined by n can be suitably used.

The salt of the biguanide-based disinfectant usable in the present invention is not particularly limited, so long as the salt is pharmacologically (pharmaceutically) or physiologically acceptable in the field of medicine. The salt of the biguanide-based disinfectant usable in the present invention includes, for example, salts of inorganic acids such as hydrochloric acid, hydrobromic acid, sulfuric acid, boric acid, phosphoric acid, and nitric acid; salts of organic acids such as acetic acid, gluconic acid, maleic acid, ascorbic acid, stearic acid, tartaric acid, and citric acid; sulfonic acid salts such as methanesulfonates, isethionates, benzenesulfonates, and p-toluenesulfonates, and salts of inorganic acids are preferred, and hydrochlorides are more preferred. These salts may be used alone, or in any combinations of two or more kinds.

In the present invention, the biguanide-based disinfectant may be used alone, or in any combinations of two or more kinds.

In the ophthalmic composition of the present invention, the content of the component (A) is appropriately set depending upon the kinds of the component (A), the pharmaceutical dosage forms of the ophthalmic composition, and the like, and the component (A) in terms of a total content is, for example, from 0.000001 to 0.01 w/v %, preferably from 0.00001 to 0.005 w/v %, and more preferably from 0.0001 to 0.001 w/v %, based on a total amount of the ophthalmic composition.

The content of the component (A) mentioned above is preferred, from the viewpoint of even more increasing a disinfectant action of Acanthamoeba, and also preferred, from the viewpoint of even more increasing an improving action of corneal barrier functions, and from the viewpoint of even more increasing an inhibitory action of size changes of soft contact lenses caused by boric acid and/or a salt thereof.

The ophthalmic composition of the present invention contains, in addition to the above-mentioned component (A), zinc chloride (which may also be hereinafter referred to as the component (B)). As described above, since the component (A) and the component (B) are included, it is made possible to remarkably increase a disinfectant action of Acanthamoeba. In addition, since the component (A) and the component (B) are included, the lowering of the corneal barrier functions induced by the above-mentioned component (A) is inhibited. Further, since the component (A) and the component (B) are included in the ophthalmic composition containing boric acid and/or a salt thereof, it is made possible to effectively inhibit size changes of contact lenses caused by boric acid and/or a salt thereof.

Zinc chloride usable in the present invention can be used without particular limitations, so long as zinc chloride can be used in the ophthalmic composition. For example, zinc chloride as prescribed in The Japanese Pharmacopeia Sixteenth Edition can be used.

Zinc chloride usable in the present invention may be in the form of a hydrate.

In the ophthalmic composition of the present invention, the content of the component (B) is appropriately set depending upon the pharmaceutical dosage forms of the ophthalmic composition, and the like, and the component (B) in terms of a total content is, for example, from 0.00005 to 0.05 w/v %, preferably from 0.0001 to 0.015 w/v %, and more preferably from 0.0005 to 0.005 w/v %, based on a total amount of the ophthalmic composition.

The content of the component (B) mentioned above is preferred, from the viewpoint of even more increasing a disinfectant action of Acanthamoeba, and the content is also preferred, from the viewpoint of even more increasing an improving action of corneal barrier functions, and from the viewpoint of even more increasing an inhibitory action of size changes of soft contact lenses caused by boric acid and/or a salt thereof.

In addition, in the ophthalmic composition of the present invention, the content proportion of the component (B) based on the component (A) is not particularly limited, and a total content of the above-mentioned component (B) is, for example, from 10 to 50,000 parts by weight, preferably from 20 to 15,000 parts by weight, more preferably from 100 to 5,000 parts by weight, and especially preferably from 300 to 3,000 parts by weight, based on 100 parts by weight of a total content of the component (A), from the viewpoint of even more increasing a disinfectant action of Acanthamoeba. The above-mentioned content proportion is preferred, from the viewpoint of even more increasing an improving action of corneal barrier functions, and from the viewpoint of even more increasing an inhibitory action of size changes of soft contact lenses caused by boric acid and/or a salt thereof.

The ophthalmic composition of the present invention may further contain, in addition to the component (A) and the component (B) mentioned above, at least one member selected from the group consisting of boric acid and salts thereof (which may also be hereinafter simply referred to as the component (C)).

Boric acid is a collective name for an oxyacid formed by hydrogenating diboron trioxide, which includes orthoboric acid, metaboric acid, tetraboric acid, and the like. Boric acid is a known compound, and can be synthesized in accordance with a known method, or can be obtained as a commercially available product.

The salt of boric acid usable in the present invention is not particularly limited, so long as the salt is pharmacologically (pharmaceutically) or physiologically acceptable in the field of medicine as the limitation. The salts of boric acid usable in the present invention include, for example, salts with organic bases, including, for example, salts with organic amines, such as methylamine, triethylamine, triethanolamine, morpholine, piperazine, pyrrolidine, amino acids, tripyridine, and picoline; salts with inorganic bases, including alkali metal salts, e.g., sodium salts, potassium salts, and the like; alkaline earth metal salts, e.g., calcium salts, magnesium salts, and the like; other metal salts, e.g. aluminum salts and the like, and the salts with inorganic bases are preferred, alkali metal salts, alkaline earth metal salts, and ammonium salts are more preferred, alkali metal salts are even more preferred, and sodium salts are especially preferred. In the present invention, the salts of boric acid may be used alone, or in any combinations of two or more kinds. In addition, the boric acid and/or a salt thereof usable in the present invention may be in the form of a hydrate.

In the ophthalmic composition of the present invention, the component (C) may be used alone, or in any combinations of two or more kinds. The component (C) usable in the present invention includes, for example, orthoboric acid, metaboric acid, tetraboric acid, sodium orthoborate, sodium metaborate, sodium tetraborate, potassium orthoborate, potassium metaborate, potassium tetraborate, ammonium orthoborate, ammonium metaborate, ammonium tetraborate, borax, and the like, and it is preferable that orthoboric acid and borax are used in combination. Here, as orthoboric acid and borax, products acceptable by The Japanese Pharmacopeia Sixteenth Edition are preferably used, and commercially available products can also be used.

In the ophthalmic composition of the present invention, when orthoboric acid and borax are used in combination as the component (C), the content proportion of the borax to the orthoboric acid is not particularly limited, and a total content of the borax is usually from 0.1 to 200 parts by weight, preferably from 1 to 100 parts by weight, and more preferably from 4 to 50 parts by weight, based on 100 parts by weight of a total content of orthoboric acid.

In the ophthalmic composition of the present invention, the content of the component (C) is appropriately set depending upon the pharmaceutical dosage forms of the ophthalmic composition and the like, and a total content of the component (C) calculated as a boron atom content is, for example, from 0.001 to 0.045 mol/100 mL, more preferably from 0.004 to 0.04 mol/100 mL, and even more preferably from 0.005 to 0.038 mol/100 mL, based on a total amount of the ophthalmic composition. Here, the content of the component (C) calculated as a boron atom content refers to a content calculated as the content of boron atoms contained in boric acid and/or a salt thereof contained in the ophthalmic composition, and the boron atom content can be easily calculated from the content of the component (C) of the ophthalmic composition. For example, when the ophthalmic composition contains, as the component (C), 0.5 w/v % of orthoboric acid and 0.02 w/v % of borax, orthoboric acid (molecular weight: 61.83) contains one atom of boron, and borax (molecular weight: 381.37) contains four atoms of boron, so that the content proportion of the boron atom originated from orthoboric acid is 0.00809 mol/100 mL, and the content of the boron atom originated from borax is 0.00021 mol/100 mL, and the content of a total amount of the component (C), calculated as a boron atom content, is 0.00830 mol/100 mL.

In addition, the component (C) is more likely to cause size changes of contact lenses; however, according to the ophthalmic composition of the present invention, in a case where the component (C) calculated as a boron atom content is 0.017 mol/100 mL or less, the size changes of the contact lenses can be even more effectively inhibited. Therefore, in the ophthalmic composition of the present invention, the content of the component (C), calculated as a boron atom content, is exemplified by from 0.0001 to 0.021 mol/100 mL, more preferably from 0.005 to 0.019 mol/100 mL, and even more preferably from 0.008 to 0.017 mol/100 mL, based on a total amount of the ophthalmic composition, from the viewpoint of even more effectively exhibiting an inhibitory action of size changes of contact lenses caused by boric acid and/or a salt thereof.

In addition, in the ophthalmic composition of the present invention, the content proportion of the component (C) mentioned above based on the above-mentioned component (A) is not particularly limited. From the viewpoint of even more increasing an inhibitory action of size changes of contact lenses in the ophthalmic composition containing boric acid and/or a salt thereof, a total content of the component (C), calculated as a boron atom content, is, for example, from 1 to 150 mol, preferably from 10 to 140 mol, and more preferably from 20 to 130 mol, based on 1 g of a total content of the component (A).

The ophthalmic composition of the present invention may further contain a buffer other than boric acid and/or a salt thereof. The buffer which can be blended in the ophthalmic composition of the present invention is not particularly limited, so long as the buffer is pharmacologically (pharmaceutically) or physiologically acceptable in the field of medicine. Other buffers which can be used in the ophthalmic composition of the present invention include, for example, phosphate buffer, carbonate buffer, citrate buffer, acetate buffer, ε-aminocaproic acid, aspartic acid, aspartates, and the like, and phosphate buffer, carbonate buffer, or citrate buffer is preferred, and phosphate buffer is more preferred. The phosphate buffer includes phosphoric acid, or phosphates such as alkali metal phosphates and alkaline earth metal phosphates. The carbonate buffer includes carbonic acid, or carbonates such as alkali metal carbonates and alkaline earth metal carbonates. The citrate buffer includes citric acid, or an alkali metal citrate, an alkaline earth metal citrate, and the like. More specific examples include phosphate buffers, such as phosphoric acid or salts thereof, e.g. disodium hydrogenphosphate, sodium dihydrogenphosphate, potassium dihydrogenphosphate, trisodium phosphate, dipotassium phosphate, calcium hydrogenphosphate, calcium dihydrogenphosphate, etc.; carbonate buffers, such as carbonic acid or salts thereof, e.g. sodium hydrogencarbonate, sodium carbonate, ammonium carbonate, potassium carbonate, calcium carbonate, potassium hydrogencarbonate, magnesium carbonate, etc.; citrate buffers, such as citric acid or salts thereof, e.g. sodium citrate, potassium citrate, calcium citrate, sodium dihydrogencitrate, disodium citrate, etc.; acetate buffers, such as acetic acid or salts thereof, e.g. ammonium acetate, potassium acetate, calcium acetate, sodium acetate, etc.; aspartic acid or aspartates, e.g. sodium aspartate, magnesium aspartate, potassium aspartate, etc. These buffers can be used alone or in any combinations of two or more kinds.

When the ophthalmic composition of the present invention is blended with a buffer other than boric acid and/or a salt thereof, the content of the buffer cannot be unconditionally determined because the content differs depending upon the kinds of buffers used, the kinds and the blending amounts of other blending components, and the pharmaceutical dosage forms of the ophthalmic composition, and the like. The content is, for example, in terms of its total content, from 0.01 to 10 w/v %, preferably from 0.1 to 5 w/v %, more preferably from 0.2 to 2 w/v %, and especially preferably from 0.2 to 1 w/v %, based on a total amount of the ophthalmic composition.

The ophthalmic composition of the present invention may further contain a surfactant. The surfactant that can be contained in the ophthalmic composition of the present invention is not particularly limited, so long as the surfactant is pharmacologically (pharmaceutically) or physiologically acceptable in the field of medicine as the limitation. The surfactant may be any one of nonionic surfactants, amphoteric surfactants, anionic surfactants, and cationic surfactants.

Specific examples of the nonionic surfactant that can be contained in the ophthalmic composition of the present invention include POE sorbitan fatty acid esters such as POE(20) sorbitan monolaurate (Polysorbate 20), POE(20) sorbitan monopalmitate (Polysorbate 40), POE(20) sorbitan monostearate (Polysorbate 60), POE(20) sorbitan tristearate (Polysorbate 65), and POE(20) sorbitan monooleate (Polysorbate 80); POE-POP block copolymers such as Poloxamer 407, Poloxamer 235, Poloxamer 188, Poloxamer 403, Poloxamer 237, and Poloxamer 124; POE hydrogenated castor oils such as POE(60) hydrogenated castor oil (polyoxyethylene hydrogenated castor oil 60); polyoxyethylene castor oils, such as Polyoxyethylene Castor Oil 3, Polyoxyethylene Castor Oil 10, Polyoxyethylene Castor Oil 20, Polyoxyethylene Castor Oil 30, Polyoxyethylene Castor Oil 35, Polyoxyethylene Castor Oil 40, Polyoxyethylene Castor Oil 50, and Polyoxyethylene Castor Oil 60; POE alkyl ethers such as POE(9) lauryl ether; POE-POP alkyl ethers such as POE(20)POP(4) cetyl ether; POE alkyl phenyl ethers such as POE(10) nonyl phenyl ethers; and the like. Here, in the compounds given above, POE stands for polyoxyethylene, POP stands for polyoxypropylene, and the numbers inside parentheses stand for the number of moles added. In addition, the amphoteric surfactants which can be contained in the ophthalmic composition of the present invention include, for example, alkyldiaminoethyl glycines or salts thereof, e.g. hydrochlorides etc. In addition, the cationic surfactants which can be contained in the ophthalmic composition of the present invention include, for example, benzalkonium chloride, benzethonium chloride, etc. Also, the anionic surfactants which can be contained in the ophthalmic composition of the present invention include, for example, alkylbenzenesulfonates, alkyl sulfates, polyoxyethylene alkyl sulfates, α-sulfomethyl esters of fatty acids, α-olefinsulfonates, etc.

The surfactant usable in the ophthalmic composition of the present invention is preferably a nonionic surfactant, more preferably POE sorbitan fatty acid esters, POE hydrogenated castor oils, or POE-POP block copolymers, and even more preferably Polysorbate 80, Polyoxyethylene Hydrogenated Castor Oil 60, or Poloxamer 407. The

POE sorbitan fatty acid esters and the POE hydrogenated castor oils are preferred, and especially the POE hydrogenated castor oils are preferred, from the viewpoint of even more increasing an inhibitory action of lowering the corneal barrier functions.

In the ophthalmic composition of the present invention, the above-mentioned surfactant may be used alone, or in a combination of two or more kinds.

When the ophthalmic composition of the present invention contains a surfactant, the content of the surfactant can be appropriately set depending upon the kinds of the surfactants, the kinds and the blending amounts of the other blending components, the pharmaceutical dosage form of the ophthalmic composition, and the like. As the content of the surfactant, the surfactant is, in terms of a total content of, for example, from 0.001 to 1.0 w/v %, preferably from 0.005 to 0.7 w/v %, and more preferably from 0.01 to 0.5 w/v %, based on a total amount of the ophthalmic composition.

In addition, the ophthalmic composition of the present invention may further contain an isotonic agent. The isotonic agent which can be contained in the ophthalmic composition of the present invention is not particularly limited, so long as the isotonic agent is pharmacologically (pharmaceutically) or physiologically acceptable in the field of medicine. The isotonic agent usable in the ophthalmic composition of the present invention includes, for example, disodium hydrogenphosphate, sodium dihydrogenphosphate, potassium dihydrogenphosphate, sodium hydrogensulfite, sodium sulfite, potassium chloride, calcium chloride, sodium chloride, magnesium chloride, potassium acetate, sodium acetate, sodium hydrogencarbonate, sodium carbonate, sodium thiosulfate, magnesium sulfate, glycerol, propylene glycol, and the like, and glycerol, propylene glycol, sodium chloride, potassium chloride, calcium chloride, or magnesium chloride is preferred, sodium chloride or glycerol is more preferred, and sodium chloride is especially preferred. In the ophthalmic composition of the present invention, the above-mentioned isotonic agent may be used alone or in any combinations of two or more kinds.

When the ophthalmic composition of the present invention contains an isotonic agent, the content of the isotonic agent cannot be unconditionally determined because the content differs depending upon the kinds of the isotonic agents used. The content of the isotonic agent in terms of a total content of the isotonic agent is, for example, from 0.01 to 10 w/v %, preferably from 0.05 to 5 w/v %, and more preferably from 0.1 to 3 w/v %, based on a total amount of the ophthalmic composition.

The pH of the ophthalmic composition of the present invention is not particularly limited, so long as the pH is pharmacologically (pharmaceutically) or physiologically acceptable in the field of medicine. The pH of the ophthalmic composition of the present invention is, for example, from 4.0 to 9.5, preferably from 5.0 to 9.0, more preferably from 6.2 to 8.5, and especially preferably from 6.5 to 8.

In addition, the osmotic pressure of the ophthalmic composition of the present invention is not particularly limited, so long as the osmotic pressure is within the range acceptable by a live body. The osmotic pressure ratio of the ophthalmic composition of the present invention is, for example, from 0.3 to 5.0, preferably from 0.4 to 3.0, and more preferably from 0.5 to 1.5. The adjustment of the osmotic pressure can be carried out by a known method in the art with an organic salt, a polyhydric alcohol, a sugar alcohol, a saccharide, or the like. The osmotic pressure ratio is defined as a ratio of an osmotic pressure of a sample to 286 mOsm (osmotic pressure of an aqueous 0.9 w/v % sodium chloride solution) as prescribed in The Japanese Pharmacopeia Sixteenth Edition, and an osmotic pressure is measured referring to a method for measuring an osmotic pressure (cryoscopic method) as prescribed in The Japanese Pharmacopeia. Here, the standard solution for measuring an osmotic pressure ratio (aqueous 0.9 w/v % sodium chloride solution) is prepared by drying sodium chloride (The Japanese Pharmacopeia standard reagent) at a temperature of from 500° to 650° C. for 40 to 50 minutes, and thereafter allowing it to cool in a desiccator (silica gel), accurately weighing 0.900 g of the cooled sample, and dissolving the sample in purified water to make up a volume of 100 mL, or alternatively, a commercially available standard solution for measuring an osmotic pressure ratio (aqueous 0.9 w/v % sodium chloride solution) is used.

The ophthalmic composition of the present invention may contain, in addition to the above-mentioned components, various pharmacologically active ingredients and physiologically active ingredients in combination in proper amounts, so long as the ingredients would not hamper the effects of the present invention. The ingredients are not particularly limited, and the ingredients are exemplified by, for example, active ingredients in the ophthalmic agent as prescribed in Ippan-yo Iyakuhin Seizo (Yunyu) Shonin Kijun 2000-nen-ban (2000 Edition, Guidelines for Acceptance of Manufacture (Import) of OTC (over-the-counter) Drugs) (Edited by Yakuji Shinsa Kenkyukai). Concrete examples of the ingredients usable in the ophthalmic agent include the following ingredients.
antihistamines: for example, iproheptine, diphenhydramine hydrochloride, chlorphenylamine maleate, ketotifen fumarate, and the like.
decongestants: for example, tetrahydrozoline hydrochloride, naphazoline hydrochloride, naphazoline sulfate, epinephrine hydrochloride, ephedrine hydrochloride, methylephedrine hydrochloride, and the like.
disinfectants: for example, cetyl pyridinium, benzalkonium chloride, benzethonium chloride, chlorhexidine hydrochloride, chlorhexidine gluconate, and the like.
vitamins: for example, flavin adenine dinucleotide sodium, cyanocobalamin, retinol acetate, retinol palmitate, pyridoxine hydrochloride, pantenol, calcium pantothenate, tocopherol acetate, and the like.
amino acids: for example, potassium aspartate, magnesium aspartate, monoethanolamine, 2-amino-2-methyl-1,3-propanediol, glycine, and the like.
antiphlogistics: for example, dipotassium glycyrrhizate, pranoprofen, allantoin, azulene, sodium azulene sulfonate, guaiazulene, ε-aminocaproic acid, berberine chloride, berberine sulfate, lysozyme chloride, licorice, and the like.
others: for example, sodium chromoglycate, sodium chondroitin sulfate, sodium hyaluronate, sulfamethoxazole, sulfamethoxazole sodium, and the like.

In addition, in the ophthalmic composition of the present invention, various additives are properly selected in accordance with a conventional method in accordance with their applications and the pharmaceutical dosage forms, and one or more additives may be contained in the composition in proper amounts, so long as the ingredients would not hamper the effects of the present invention. As additives thereof, for example, various additives listed in Pharmaceutical Additive Dictionary 2007 (edited by Association of Japan Pharmaceutical Additives) can be exemplified. The representative ingredients include the following additives.
carriers: for example, aqueous carriers such as water and water-containing ethanol.
sugars: for example, cyclodextrin, and the like.
sugar alcohols: for example, xylitol, sorbitol, mannitol, and the like, wherein these may be in d-form, 1-form, or dl-form.
antiseptics, disinfectants, or antibacterial agents: for example, alkyldiaminoethylglycine hydrochloride, sodium benzoate, ethanol, benzalkonium chloride, benzethonium chloride, chlorhexidine gluconate, chlorobutanol, sorbic acid, potassium sorbate, sodium dehydroacetate, methyl parahydroxybenzoate, ethyl parahydroxybenzoate, propyl parahydroxybenzoate, butyl parahydroxybenzoate, oxyquinoline sulfate, phenethyl alcohol, benzyl alcohol, Glokill (trade name, manufactured by Rhodia Co., Ltd.), and the like.
pH adjusting agents: for example, hydrochloric acid, ε-aminocaproic acid, citric acid, acetic acid, sodium hydroxide, potassium hydroxide, calcium hydroxide, magnesium hydroxide, sodium hydrogencarbonate, sodium carbonate, triethanolamine, monoethanolamine, diisopropanolamine, sulfuric acid, phosphoric acid, polyphosphoric acid, propionic acid, oxalic acid, gluconic acid, fumaric acid, lactic acid, tartaric acid, malic acid, succinic acid, gluconolactone, ammonium acetate, and the like.
stabilizers: for example, dibutylhydroxytoluene, trometamol, sodium formaldehyde sulfoxylate (Rongalit), tocopherol, sodium pyrosulfite, monoethanolamine, aluminum monostearate, glycerol monostearate, and the like.
chelating agents: for example, ethylenediaminediacetic acid (EDDA), ethylenediaminetriacetic acid, ethylenediaminetetraacetic acid (edetic acid, EDTA), N-(2-hydroxyethyl)ethylenediaminetriacetic acid (HEDTA), diethylenetriaminepentaacetic acid (DTPA), and the like.

The ophthalmic composition of the present invention is prepared by adding a component (A) and a component (B) mentioned above, and optionally other blending ingredients so as to have desired blending proportions.

The ophthalmic composition of the present invention is not particularly limited in its dosage form, so long as the ophthalmic composition can be used in the ophthalmic field, and the ophthalmic composition includes, liquids, ointments, and the like. Among them, liquids are preferred. In addition, among the liquids, aqueous liquids are preferred. When the ophthalmic composition is in the form of an aqueous liquid, water which is pharmacologically (pharmaceutically) or physiologically acceptable in the field of medicine may be used as an aqueous carrier. Concretely, the water as mentioned above is exemplified by distilled water, tap water, purified water, sterilized purified water, injection water, injection distilled water, and the like. These definitions are as prescribed in Japanese Pharmacopeia Sixteenth Edition. Here, the aqueous liquid means the form of a liquid containing water, and usually, water is contained in an amount of 1% by weight or more, preferably 5% by weight or more, more preferably 20% by weight or more, and even more preferably 50% by weight or more, of the ophthalmic composition.

In addition, the aqueous composition means usually a composition containing water in an amount of 1% by weight or more, preferably 5% by weight or more, more preferably 20% by weight or more, and even more preferably 50% by weight or more, of the composition. Concretely, the water used in the aqueous composition is exemplified by distilled water, tap water, purified water, sterilized purified water, injection water, injection distilled water, and the like, in the same manner as above.

Therefore, the present invention provides a method for producing an ophthalmic composition, including adding (A) a biguanide-based disinfectant and (B) zinc chloride to a carrier containing water, from another viewpoint.

The ophthalmic composition of the present invention is not limited to the pharmaceutical dosage forms, so long as the ophthalmic composition is usable in the ophthalmic field. The pharmaceutical dosage forms include, for example, eye drops, including eye drops for contact lenses; eyewashes, including eyewashes for contact lenses; contact lens wearing solutions; agents for contact lens care, including disinfectant solutions for contact lens, storage solutions for contact lens, cleansing solutions for contact lenses, cleansing-storage solutions for contact lenses, and the like; disinfection-cleansing-storage solutions for contact lenses, including multi-purpose solutions, and the like. The ophthalmic composition of the present invention can inhibit the lowering of the corneal barrier functions induced when a biguanide-based disinfectant is used, the ophthalmic composition can be used even in pharmaceutical dosage forms that are directly applied to the corneal surface, including eye drops, including eye drops for contact lenses, eyewashes, including eye washes for contact lenses, contact lens wearing solutions, storage solutions for contact lens, cleansing-storage solutions for contact lenses, disinfection-cleansing-storage solutions for contact lenses, and the like, with high safety.

In addition, the Acanthamoeba corneal infectious diseases are suffered most by individuals wearing contact lenses, so that an ophthalmic composition for contact lenses having a disinfectant action against Acanthamoeba is strongly in demand. Therefore, preferred pharmaceutical dosage forms of the ophthalmic composition of the present invention include ophthalmic compositions for contact lenses, such as eye drops for contact lenses, i.e. eye drops which can be used while wearing contact lenses on the eyes; eyewashes for contact lenses, i.e. eyewashes which can be used while wearing contact lenses on the eyes; contact lens wearing solutions; agents for contact lens care, and the like. Among them, it is very important for the agents for contact lens care to disinfect the contact lenses against Acanthamoeba, which are the preferred pharmaceutical dosage form in the ophthalmic composition of the present invention. In addition, as to the disinfection-cleansing-storage solutions for contact lenses, e.g. multi-purpose solutions, since the contact lenses go through the steps of disinfection, cleansing, and storage all-in-one solution, the solutions are formulations that directly contact the cornea via contact lenses. Therefore, the kinds of the disinfectants to be blended and the concentrations thereof are inevitably limited to a range that would not impair safety, and it is important to abide by the methods of use such as disinfection time or rub-cleaning in order to obtain the sufficient disinfectant strength. However, in actuality, the methods of use may not be strictly abided thereby, whereby consequently sufficient disinfectant strength cannot be obtained. Therefore, it is very important for the disinfection-cleansing-storage solutions for contact lenses that Acanthamoeba deposited on the contact lenses is sufficiently disinfected to prevent Acanthamoeba corneal infectious diseases, and the solutions are especially suitable as the pharmaceutical dosage forms of the ophthalmic composition.

When the ophthalmic composition of the present invention is used as an ophthalmic composition for contact lenses, the kinds of the contact lenses to which the ophthalmic composition is applied are not particularly limited, and the ophthalmic composition can be applied to all the contact lenses that are presently commercially available or would be commercially available in future, embracing any of soft contact lenses, hard contact lenses, and oxygen-permeable hard contact lenses. In addition, the soft contact lenses embrace both silicone hydrogel contact lenses and non-silicone hydrogel contact lenses, i.e. soft contact lenses that are not silicone hydrogel lenses as subjects to which the ophthalmic composition is applied. The Acanthamoeba corneal infectious diseases are suffered most by individuals wearing soft contact lenses among the individuals wearing contact lenses, so that an ophthalmic composition capable of exhibiting high disinfection effects against Acanthamoeba for soft contact lenses is strongly in demand. In addition, among the soft contact lenses, it is said to have a relatively high polluting ratio of Acanthamoeba in soft contact lenses in which a water content is 50% or more and monomers having an anion are contained in an amount of 1% by mol or more of the constituting monomers of the raw material polymer, i.e. soft contact lenses of which soft contact lens classification is Group IV, and contact lenses in which silicone is blended with hydrogel, i.e. silicone hydrogel contact lenses. In consideration of the above viewpoints, the preferred subject to which the ophthalmic composition of the present invention is applied includes soft contact lenses, and more preferred examples include soft contact lenses in which a water content is 50% or more and monomers having an anion are contained in an amount of 1% by mol or more of the constituting monomers of the raw material polymer, and silicone hydrogel contact lenses.

Here, the soft contact lens classifications as used herein are classifications as prescribed in “Method for Classification of Soft Contact Lenses” defined in “Handling and the like of Materials to Be Attached to the Petition for Acceptance of Manufacture (Import) of Soft Contact Lenses and Disinfectants for Soft Contact Lenses” notified as lyakushin (Examination Division of Pharmaceutical Affairs Bureau) No. 645 dated Mar. 31, 1999, issued by Chief of Evaluation and Licensing Division, Pharmaceutical Safety Bureau, the Ministry of Health, Labor and Welfare, and the present classification complies with the soft contact lens classification method as prescribed by FDA (U.S. Federal Food and Drug Administration).

Here, the water content of the contact lenses refers to a proportion of water in the contact lenses, which is concretely calculated by the following calculation formula.

$$Water Content ( \% ) = \frac{Weight of Water Contained}{Weight of Contact Lens in Water \text{-} Containing State} \times 100$$

The water content ratio can be measured in accordance with a method for measuring weight in accordance with the description of IS018369-4:2006.

In addition, it is said that the silicone hydrogel contact lenses are likely to cause an ectocorneal disorder called corneal staining, when the contact lenses are treated with an ophthalmic composition containing a biguanide-based disinfectant. Therefore, according to the present invention, since the composition contains zinc chloride together with a biguanide-based disinfectant, the corneal barrier functions are improved, so that the ectocorneal disorders can be effectively inhibited. In view of the above effects of the present invention, one embodiment of the preferred subject to which the ophthalmic composition of the present invention is applied includes silicone hydrogel contact lenses.

In addition, on the other hand, it is said that the soft contact lenses of which soft contact lens classification is Group IV are relatively likely to cause the expansion of lenses when treated for a long period of time in an ophthalmic composition containing boric acid and/or a salt thereof, among which the soft contact lenses of Group IV produced by materials containing methacrylic acid monomers are confirmed to show strong likeliness thereof. The materials containing the methacrylic acid monomers mentioned above include, as the names of materials as prescribed in the USAN (United States Adopted Names), for example, Etafilcon A, Ocufilcon A, Ocufilcon B, Ocufilcon C, Ocufilcon D, Ocufilcon E, Methafilcon A, Methafilcon B, Focofilcon A, Perfilcon A, Vifilcon A, Vifilcon B, and the like, and the ophthalmic composition of the present invention is suitably applied to soft contact lenses made of these materials. The soft contact lenses to which the composition is applied in the present invention more preferably include soft contact lenses of Group IV made of Etafilcon A, Methafilcon A, and Methafilcon B as materials, and especially preferably include soft contact lenses of Group IV made of Etafilcon A as a material. In addition, the soft contact lenses which are subject to be applied in the present invention are preferably lenses of which surfaces are charged negatively.

The ophthalmic composition of the present invention is used in accordance with the method of use according to the pharmaceutical dosage forms thereof. For example, when the ophthalmic composition of the present invention is in the pharmaceutical dosage forms of the agents for contact lens care, it is desired that contact lenses are immersed in an agent for contact lenses for 1 hour or more, and preferably 4 hours or more, in order to sufficiently exhibit disinfection effects of Acanthamoeba. In addition, in a case where the ophthalmic composition of the present invention is in the pharmaceutical dosage form of eye drops, including eye drops for contact lenses, or eyewashes, including eyewashes for contact lenses, the ophthalmic composition can be directly dropped to or subjected to eye-washing naked eyes or eyes wearing contact lenses, or the ophthalmic composition can be dropped to eyes before wearing contact lenses.

The ophthalmic composition of the present invention can exhibit an excellent disinfection action against Acanthamoeba, so that the ophthalmic composition can be used in the applications of preventing Acanthamoeba corneal infectious diseases.

In addition, the ophthalmic composition of the present invention can inhibit the lowering of corneal barrier functions induced when a biguanide-based disinfectant is used, so that the cornea can be maintained in a healthy state, thereby making it useful as a prophylactic agent for the ectocorneal disorders.

The ophthalmic composition for contact lenses of the present invention can be provided by housing the ophthalmic composition in any sorts of containers. The container for housing the ophthalmic composition for contact lenses of the present invention is not particularly limited, and any container made of materials that can be used in a general container in the field of the art may be used, and, for example, glass materials and plastic materials, e.g. polyethylene terephthalate resin, polypropylene resin, polyethylene resin, polyethylene naphthalate resin, and the like, can be properly selected and used according to their purposes and applications. In addition, the container for housing the ophthalmic composition for contact lenses of the present invention may be a transparent container in which the internal of the container can be visually recognized, or an opaque container which is difficult to visually recognize the internal. Since the confirmation of the amount of the solution of the ophthalmic composition, foreign objects tests during the production steps, and the like are facilitated, especially transparent containers are preferred. Here, the term “transparent container” includes both colorless transparent containers and colored transparent containers.

In addition, the present invention provides use of (A) a biguanide-based disinfectant and (B) zinc chloride in the manufacture of an ophthalmic composition, from a different viewpoint.

Further, the present invention provides use of a composition containing (A) a biguanide-based disinfectant and (B) zinc chloride as an ophthalmic composition, from a different viewpoint.

Further, the present invention provides a composition containing (A) a biguanide-based disinfectant and (B) zinc chloride, for use in an ophthalmic composition, from a different viewpoint.

2. Anti-Acanthamoeba Agent

As mentioned above, since the agent contains a biguanide-based disinfectant and zinc chloride, a disinfectant action against Acanthamoeba can be enhanced and exhibited.

Therefore, the present invention provides an anti-Acanthamoeba agent containing (A) a biguanide-based disinfectant and (B) zinc chloride, from another different viewpoint.

The present anti-Acanthamoeba agent is added to various compositions and used in order to give a disinfectant action against Acanthamoeba. In the present anti-Acanthamoeba agent, the kinds, the contents, and the content proportions of the component (A) and the component (B) are the same as those of “1. Ophthalmic Composition” mentioned above.

The composition to which the present anti-Acanthamoeba agent is added is not particularly limited, and the composition is preferably exemplified by an ophthalmic composition. The kinds, the contents, the content proportions, and the blending proportions of the present anti-Acanthamoeba agent added to various compositions are properly set depending upon the kinds of the compositions. For example, in a case where the present anti-Acanthamoeba agent is added to an ophthalmic composition and used, the kinds, the contents, the content proportions, and the blending proportions of the agent added to the ophthalmic composition, and the pharmaceutical dosage forms of the ophthalmic composition to be added, and the like are the same as those of “1. Ophthalmic Composition” mentioned above.

In these methods and compositions, the component (A) and the component (B) may be co-present in an ophthalmic composition for contact lenses, and the orders of addition thereof are not particularly limited.

3. Method for Disinfecting Against Acanthamoeba, Method for Enhancing Disinfectant Action Against Acanthamoeba, and Method for Giving Disinfectant Action Against Acanthamoeba to Ophthalmic Composition

As mentioned above, since a biguanide-based disinfectant and zinc chloride are contained, a disinfectant action against Acanthamoeba can be enhanced and exhibited.

Therefore, the present invention provides a method for disinfecting against Acanthamoeba, characterized by disinfecting against Acanthamoeba with an ophthalmic composition containing (A) a biguanide-based disinfectant, and (B) zinc chloride, from another different viewpoint. Also, the present invention provides a method for enhancing a disinfectant action against Acanthamoeba of the ophthalmic composition, characterized by blending (B) zinc chloride in an ophthalmic composition containing (A) a biguanide-based disinfectant. Further, the present invention also provides a method for giving a disinfectant action against Acanthamoeba to an ophthalmic composition, characterized by blending (A) a biguanide-based disinfectant and (B) zinc chloride in an ophthalmic composition.

In these methods, the kinds, the contents, the content proportions, and the blending proportions of the component (A) and the component (B) used, and the kinds, the contents, the content proportions, and the blending proportions of the other components to be blended, and the pharmaceutical dosage forms of the ophthalmic composition, and the like are the same as those of “1. Ophthalmic Composition” mentioned above.

In addition, in these methods, the component (A) and the component (B) may be co-present in an ophthalmic composition for contact lenses, and orders of addition thereof are not particularly limited.

4. Method for Improving Corneal Barrier Functions, and Method for Giving Action of Improving Corneal Barrier Functions to Ophthalmic Composition

As mentioned above, corneal barrier functions can be improved by including a biguanide-based disinfectant and zinc chloride in an ophthalmic composition.

Therefore, the present invention provides a method for preparing an ophthalmic composition having improved corneal barrier functions, characterized by blending (A) a biguanide-based disinfectant and (B) zinc chloride in an ophthalmic composition, from another different viewpoint.

In addition, the present invention provides a method for giving an action of improving corneal barrier functions to an ophthalmic composition, characterized by blending (B) zinc chloride in an ophthalmic composition containing (A) a biguanide-based disinfectant.

In these methods, the kinds, the contents, the content proportions, and the blending proportions of the component (A) and the component (B) used, and the kinds, the contents, the content proportions, and the blending proportions of the other components to be blended, and the pharmaceutical dosage forms of the ophthalmic composition, and the like are the same as those of “1. Ophthalmic Composition” mentioned above.

In addition, in these methods, the component (A) and the component (B) may be co-present in an ophthalmic composition for contact lenses, and orders of addition thereof are not particularly limited.

5. Method for Inhibiting Size Changes of Contact Lenses Caused by Boric Acid and/or Salt Thereof, and Method for Giving Action of Inhibiting Size Changes of Contact Lenses Caused by Boric Acid and/or Salt Thereof to Ophthalmic Composition

As mentioned above, size changes of the contact lenses caused by bringing into contact with boric acid and/or a salt thereof can be inhibited by including a biguanide-based disinfectant and zinc chloride in an ophthalmic composition.

Therefore, the present invention provides a method for inhibiting size changes of contact lenses caused by boric acid and/or a salt thereof, characterized by blending (A) a biguanide-based disinfectant and (B) zinc chloride in an ophthalmic composition containing (C) at least one member selected from the group consisting of boric acid and salts thereof, from another different viewpoint.

In addition, the present invention provides a method for giving an action of inhibiting size changes of contact lenses caused by boric acid and/or a salt thereof to an ophthalmic composition, characterized by blending (A) a biguanide-based disinfectant and (B) zinc chloride in an ophthalmic composition containing (C) at least one member selected from the group consisting of boric acid and salts thereof.

In these methods, the contents, the content proportions, and the blending proportions of (C) boric acid and/or a salt thereof, the kinds, the contents, the content proportions, and the blending proportions of the component (A) and the component (B), and the kinds, the contents, the content proportions, and the blending proportions of the other components to be blended, and the pharmaceutical dosage forms of the ophthalmic composition, and the like are the same as those of “1. Ophthalmic Composition” mentioned above.

In addition, in these methods, the component (A), the component (B), and the component (C) may be co-present in an ophthalmic composition for contact lenses, and addition orders thereof are not particularly limited.

### EXAMPLES

The present invention will be described in detail hereinbelow by means of Examples, without intending to limit the present invention to these Examples.

### Test Example 1

### Evaluation (1) of Disinfectant Effects Against Acanthamoeba

Each of the ophthalmic compositions was prepared in accordance with the following Table 1, and the disinfectant effects of each ophthalmic composition against Acanthamoeba (Acanthamoeba castellanli, ATCC 30868) were evaluated.

The cultured Acanthamoeba was washed three times with isotonic phosphate buffered saline (PBS) [Dulbecco PBS(−)powder “NISSUI” (manufactured by NISSUI PHARMACEUTICAL CO., LTD.); containing 0.8 w/v % sodium chloride, 0.02 w/v % potassium chloride, 0.115 w/v % sodium hydrogenphosphate, 0.02 w/v % potassium dihydrogenphosphate], and thereafter suspended in the above-mentioned PBS solution so as to have a concentration of 5.0×10⁶ cells/mL, to prepare an Acanthamoeba suspension. Next, 100 μL of the above-mentioned Acanthamoeba suspension was inoculated to each of the ophthalmic compositions (Examples 1 to 3 and Comparative Example 1) so as to have a final concentration of 5.0×10⁴ cells/mL as a test solution, and allowed to stand at 25° C. In addition, as a control solution, a solution adjusted with the above-mentioned PBS solution so as to have a final concentration of 5.0×10⁴ cells/mL was furnished, and allowed to stand at 25° C. in the same manner. After 4 hours, each of the test solutions and the control solution was subjected to a neutralization treatment by adding a neutralizing solution (Dey-Engley Neutralizing Broth). Thereafter, the remaining number of Acanthamoeba was counted, and a reduction in the remaining number of Acanthamoeba after the test from the number of Acanthamoeba at the beginning of the test on a logarithmic scale (log reduction) was calculated.

TABLE 1

| | Comp. | | | |
|---|---|---|---|---|
| | Ex. 1 | Ex. 1 | Ex. 2 | Ex. 3 |
| Zinc Chloride | — | 0.00015 | 0.0015 | 0.015 |
| PHMB¹⁾ | 0.0001 | 0.0001 | 0.0001 | 0.0001 |
| | (1 ppm) | (1 ppm) | (1 ppm) | (1 ppm) |
| Purified Water | Bal. | Bal. | Bal. | Bal. |

Units: w/v %.
¹⁾“PHMB”: Hydrochloride of polyhexamethylene biguanide (compound represented by the general formula (II), wherein n is 12 on average), was used.

The results are shown in FIG. 1. As is clear from FIG. 1, the ophthalmic compositions containing PHMB and zinc chloride completely unexpectedly had a remarkable reduction in Acanthamoeba, as compared to the ophthalmic composition containing PHMB alone (Comparative Example 1), so that it was clarified that the disinfectant effects against Acanthamoeba were remarkably increased by including PHMB and zinc chloride (Examples 1 to 3).

### Referential Test Example 1

### Evaluation (2) of Disinfectant Effects Against Acanthamoeba

In order to confirm that whether or not the disinfectant effects against Acanthamoeba are exhibited even when zinc chloride is used alone, the following experiment was conducted.

Concretely, a test was conducted in the same manner as in the above-mentioned Test Example 1, except that an ophthalmic composition as listed in the following Table 2 was used to evaluate the disinfectant effects against Acanthamoeba.

TABLE 2

| | Comp. | Comp. |
|---|---|---|
| | Ex. 2 | Ex. 3 |
| Zinc Chloride | 0.0015 | 0.015 |
| PHMB¹⁾ | — | — |
| Purified Water | Bal. | Bal. |

Units: w/v %.
¹⁾“PHMB”: Hydrochloride of polyhexamethylene biguanide (compound represented by the general formula (II), wherein n is 12 on average), was used.

The results are shown in FIG. 2. As shown in FIG. 2, in the ophthalmic compositions containing zinc chloride alone (Comparative Examples 2 and 3), the reduction in Acanthamoeba was not seen, as found in the above-mentioned Test Example 1, hardly observing any disinfectant effects against Acanthamoeba. It was clarified from the above that high disinfectant effects against Acanthamoeba are shown by synergistic actions between zinc chloride and the biguanide-based disinfectant.

### Test Example 2

### Evaluation (1) of Corneal Barrier Functions

Each of the ophthalmic compositions was prepared in accordance with the following Table 3, and the influences on the corneal barrier functions were evaluated.

Human ectocorneal cell line HCE-T was seeded inside a culture-insert of Transwell (24-well, Corning) so as to have a proportion of 1.0×10⁵ cells/well. The wells were dispensed with 600 μL of a culture medium, and the cells were cultured under the conditions of a temperature of 37° C. and 5% CO₂ for 6 days. After the culture, the culture medium inside the culture-insert was removed by suction, the cells were washed once with saline, and 200 μL of each ophthalmic composition was then added to the culture-insert. The culture-insert was transferred to the wells dispensed with 600 μL of each ophthalmic composition, and allowed to stand at room temperature for 10 minutes. After the treatment, each of the ophthalmic compositions inside the culture-insert was removed by suction, the cells were washed once with saline, and thereafter the culture-insert was dispensed with 200 μL of saline. The culture-insert was transferred to the wells dispensed with 1 mL of saline, and allowed to stand at room temperature for 10 minutes. Using MILLICELL(registered trademark)-ERS (manufactured by MILLIPORE CORPORATION), transepithelial electric resistance value (hereinafter also indicated as TER value) was measured. An increase ratio of TER value was calculated using the TER value obtained in accordance with the following formula (1). Here, in Test Example 2, the corresponding comparative example in the formula is Comparative Example 4.

$$Increase Ratio ( \% ) of TER Value = \frac{ \begin{matrix}TER Value of Each of \\ Comparative Examples and Examples\end{matrix} }{TER Value of Corresponding Comparative Example} \times 100 \qquad formula ( 1 )$$

Next, the saline inside the culture-insert was removed by suction, and the culture-insert was dispensed with 100 μL of a 0.01% by weight fluorescein sodium solution prepared with saline. The culture-insert was transferred to wells dispensed with 600 μL of the saline, and allowed to stand at room temperature for 20 minutes. One-hundred microliters of the saline in the wells were transferred to a 96-well microplate, and a fluorescent value was measured with a fluorescent plate reader (Fluoroskan Ascent CF, manufactured by Labsystems) at an excitation wavelength 485 nm/emitted wavelength 527 nm.

TABLE 3

| | Comp. | |
|---|---|---|
| | Ex. 4 | Ex. 4 |
| Zinc Chloride | — | 0.0025 |
| Orthoboric Acid²⁾ | 1.0 | 1.0 |
| Borax | 0.25 | 0.25 |
| Sodium Chloride | 0.7 | 0.7 |
| PHMB¹⁾ | 0.0001 | 0.0001 |
| | (1 ppm) | (1 ppm) |
| Hydrochloric Acid | q.s. | q.s. |
| Purified Water | Bal. | Bal. |
| pH | 7.0 | 7.0 |

Units of Blending Proportions: w/v %.
¹⁾“PHMB”: Hydrochloride of polyhexamethylene biguanide (compound represented by the general formula (II), wherein n is 12 on average), was used.
²⁾“Orthoboric Acid” is accepted as the standard of “boric acid” by The Japanese Pharmacopeia Sixteenth Edition.

The results obtained are shown in FIG. 3. The fluorescent value reflects the fluorescein amount transmitted through the ectocorneal cell layer. The higher the fluorescent values, the lower the barrier functions of the ectocorneal cells. In addition, the higher the value of the transepithelial electric resistance value (TER), the more firm the barrier functions. As is clear from FIG. 3, the ophthalmic composition containing PHMB alone (Comparative Example 4) not only showed a high fluorescent value but also a low TER value. On the other hand, it was clarified that the ophthalmic composition containing zinc chloride together with PHMB (Example 4) had a significantly lower fluorescent value than Comparative Example 4, and at the same time had a remarkable increase in a TER value, so that the corneal barrier functions were remarkably improved. The tendency of improvement in these corneal barrier functions was especially remarkable when a borate buffer, i.e. a combination of orthoboric acid and borax, was used as a buffer.

Therefore, it was clarified that the ophthalmic composition containing zinc chloride together with PHMB not only had high disinfectant effects against Acanthamoeba, but also had high improvement effects for the corneal barrier functions. Therefore, it was clarified that there is a high probability of obtaining a useful ophthalmic composition having very high prophylactic effects against the Acanthamoeba corneal infectious diseases.

### Test Example 3

### Evaluation (2) of Corneal Barrier Functions

Each of the ophthalmic compositions was prepared in accordance with the following Table 4, and the influences on the corneal barrier functions were evaluated.

Human ectocorneal cell line HCE-T was seeded inside a culture-insert of Transwell (24-well, Corning) so as to have a proportion of 1.0×10⁵ cells/well. The wells were dispensed with 600 μL of a culture medium, and the cells were cultured under the conditions of a temperature of 37° C. and 5% CO₂ for 5 days. After the culture, the culture medium inside the culture-insert was removed by suction, and the cells were washed once with 200 μL of a 0.2 M phosphate buffer, prepared with monosodium dihydrogenphosphate and disodium hydrogenphosphate, and the culture-insert was then dispensed with 200 μL of each test solution. The culture-insert was transferred to the wells dispensed with 600 μL of each ophthalmic composition, and allowed to stand at room temperature for 10 minutes. Next, each of the ophthalmic compositions inside the culture-insert was removed by suction, the cells were washed once with 200 μL of a 0.2 M phosphate buffer, and thereafter the culture-insert was dispensed with 100 μL of a 0.01% by weight fluorescein sodium solution prepared with a 0.2 M phosphate buffer. The culture-insert was transferred to the wells dispensed with 600 μL of a 0.2 M phosphate buffer, and allowed to stand at room temperature for 20 minutes. One-hundred microliters of a phosphate buffer in the well were transferred to a 96-well microplate, and a fluorescent value was measured with a fluorescent plate reader (Fluoroskan Ascent CF, manufactured by Labsystems) at an excitation wavelength 485 nm/emitted wavelength 527 nm. Next, the fluorescent values of Example 5 and Comparative Example 5 when the fluorescent value of the control was assumed to be 100% were calculated.

TABLE 4

| | | | Comp. |
|---|---|---|---|
| | Control | Ex. 5 | Ex. 5 |
| Zinc Chloride | — | 0.002 | — |
| Zinc Sulfate | — | — | 0.002 |
| Orthoboric Acid²⁾ | 1.0 | 1.0 | 1.0 |
| Borax | 0.08 | 0.08 | 0.08 |
| PHMB¹⁾ | 0.0001 | 0.0001 | 0.0001 |
| | (1 ppm) | (1 ppm) | (1 ppm) |
| Purified Water | Bal. | Bal. | Bal. |

Units of Blending Proportions: w/v %.
¹⁾“PHMB”: Hydrochloride of polyhexamethylene biguanide (compound represented by the general formula (II), wherein n is 12 on average), was used.
²⁾“Orthoboric Acid” is accepted as the standard of “boric acid” by The Japanese Pharmacopeia Sixteenth Edition.

The results are shown in FIG. 4. As is clear from FIG. 4, it was clarified that the ophthalmic composition containing zinc chloride together with PHMB had a low fluorescent value and remarkably improved the corneal barrier functions, as compared to the ophthalmic composition containing PHMB alone (control). However, in the case where zinc sulfate was used, it was clarified that particularly marked improvement effects for the corneal barrier functions were not obtained.

### Test Example 4

### Evaluation (1) of Lens Sizes

Each of the ophthalmic compositions was prepared in accordance with the following Table 5, and the size changes of soft contact lenses were evaluated.

As the pretreatment of the test for soft contact lenses, one piece each of brand-new soft contact lenses (trade name: 2-Week Acuvue (Group IV, USAN: Etafilcon A, main constituting monomers: 2-HEMA [hydroxyethyl methacrylate] and MAA [methacrylic acid], manufactured by Johnson & Johnson)) was soaked in 3 mL of saline in a 12-well microplate, and allowed to stand for 18 hours. After 18 hours, each lens was transferred to cells filled with the saline, the cells were set in a universal projector (manufactured article name: PROFILE PROJECTOR V-12B, manufactured by NIKON), and a diameter (the start value) of each lens was measured. Next, the soft contact lenses were immersed in each of the ophthalmic compositions listed in Table 5, and allowed to stand for 18 hours. After 18 hours, the cells were prewashed with each test solution, and those cells were filled with each of the ophthalmic compositions into which each lens was immersed. Subsequently, the cells were set in the universal projector, and a diameter of the lens (value after storage) was measured. The size changing ratio of each of the lenses was calculated with the following formula (2), from the value after storage of the measured diameter of the lenses. Here, all the tests were carried out indoors of which room temperature was set at 28° C.

$$Lens Size Changing Ratio ( \% ) = \frac{( Value After Storage ) - ( Start Value )}{Start Value} \times 100 \qquad formula ( 2 )$$

TABLE 5

| | Comp. | Comp. | Comp. | | |
|---|---|---|---|---|---|
| | Ex. 6 | Ex. 7 | Ex. 8 | Ex. 6 | Ex. 7 |
| Zinc Chloride | — | — | — | 0.005 | 0.005 |
| PHMB¹⁾ | — | 0.0001 | 0.0003 | 0.0001 | 0.0003 |
| | | (1 ppm) | (3 ppm) | (1 ppm) | (3 ppm) |
| Sodium | 0.7 | 0.7 | 0.7 | 0.7 | 0.7 |
| Chloride | | | | | |
| Orthoboric | 0.5 | 0.5 | 0.5 | 0.5 | 0.5 |
| Acid²⁾ | | | | | |
| Borax | 0.022 | 0.022 | 0.022 | 0.024 | 0.024 |
| Purified Water | Bal. | Bal. | Bal. | Bal. | Bal. |
| pH | 7.0 | 7.0 | 7.0 | 7.0 | 7.0 |
| Osmotic | 1.0 to | 1.0 to | 1.0 to | 1.0 to | 1.0 to |
| Pressure | 1.1 | 1.1 | 1.1 | 1.1 | 1.1 |
| Ratio | | | | | |

Units of Blending Proportions: w/v %.
¹⁾“PHMB”: Hydrochloride of polyhexamethylene biguanide (compound represented by the general formula (II), wherein n is 12 on average), was used.
²⁾“Orthoboric Acid” is accepted as the standard of “boric acid” by The Japanese Pharmacopeia Sixteenth Edition.

The results are shown in FIG. 5. As is clear from the results of Comparative Example 6, the soft contact lenses immersed in the ophthalmic composition only containing a borate buffer were found to have marked expansion (Comparative Example 6). In addition, the soft contact lenses immersed in the ophthalmic composition containing a borate buffer and PHMB (Comparative Example 7 or 8) had smaller size changes of the lenses and slight tendency of inhibiting expansion of lenses, but the effects as such were small, as compared to those of Comparative Example 6. On the other hand, the size changes of the soft contact lenses immersed in the ophthalmic compositions containing zinc chloride together with PHMB (Examples 6 and 7) were significantly lowered, as compared to those of Comparative Examples 6 to 8, and the expansion of the soft contact lenses caused by a borate buffer could be markedly inhibited.

### Test Example 5

### Evaluation (2) of Lens Sizes

Each of the test solutions containing a high concentration of a borate buffer was prepared in accordance with the following Table 6. The test was carried out in the same manner as in the above Test Example 4, except that the test solution used was changed, and the size changes of soft contact lenses were evaluated.

TABLE 6

| | Comp. | Comp. | Comp. | | |
|---|---|---|---|---|---|
| | Ex. 9 | Ex. 10 | Ex. 11 | Ex. 8 | Ex. 9 |
| Zinc Chloride | — | — | — | 0.005 | 0.005 |
| PHMB¹⁾ | — | 0.0001 | 0.0003 | 0.0001 | 0.0003 |
| | | (1 ppm) | (3 ppm) | (1 ppm) | (3 ppm) |
| Sodium | 0.5 | 0.5 | 0.5 | 0.5 | 0.5 |
| Chloride | | | | | |
| Orthoboric | 1.0 | 1.0 | 1.0 | 1.0 | 1.0 |
| Acid²⁾ | | | | | |
| Borax | 0.072 | 0.072 | 0.072 | 0.080 | 0.080 |
| Purified Water | Bal. | Bal. | Bal. | Bal. | Bal. |
| pH | 7.0 | 7.0 | 7.0 | 7.0 | 7.0 |
| Osmotic | 1.0 to | 1.0 to | 1.0 to | 1.0 to | 1.0 to |
| Pressure | 1.1 | 1.1 | 1.1 | 1.1 | 1.1 |
| Ratio | | | | | |

Units of Blending Proportions: w/v %.
¹⁾“PHMB”: Hydrochloride of polyhexamethylene biguanide (compound represented by the general formula (II), wherein n is 12 on average), was used.
²⁾“Orthoboric Acid” is accepted as the standard of “boric acid” by The Japanese Pharmacopeia Sixteenth Edition.

The results are shown in FIG. 6. As is clear from FIG. 6, even in a case where the concentration of a borate buffer was set at 1.072 w/v % (calculated as borate atom: 0.017 mol/100 mL), even more remarkable effects of inhibiting lens size changes are exhibited by the copresence of PHMB and zinc chloride (Examples 8 to 9), as compared to the ophthalmic composition containing only a borate buffer, and the ophthalmic compositions containing a borate buffer and PHMB (Comparative Examples 9 to 11).

### Test Example 6

### Evaluation (3) of Corneal Barrier Functions

The ophthalmic compositions were prepared in accordance with the following Table 7, and the influences on the corneal barrier functions were evaluated.

Human ectocorneal cell line HCE-T was seeded inside a culture-insert of Transwell (24-well, Corning) so as to have a proportion of 1.0×10⁵ cells/well. The wells were dispensed with 600 μL of a culture medium, and the cells were cultured under the conditions of a temperature of 37° C. and 5% CO₂ for 24 hours. After the culture, the culture-insert was transferred to the wells dispensed with 600 μL of each ophthalmic composition, and allowed to stand at room temperature for 10 minutes. After the treatment, the culture-insert was transferred to the wells dispensed with 600 μL of a phosphate buffer, and washed once. Thereafter, using MILLICELL(registered trademark)-ERS (manufactured by MILLIPORE CORPORATION), the transepithelial electric resistance value (TER value) was measured. The increase ratio was calculated on the basis of the formula (1). Here, concretely, the comparative example corresponding to Examples 10 to 12 is Comparative Example 12, the comparative example corresponding to Examples 13 and 14 is Comparative Example 13, and the comparative example corresponding to Example 15 is Comparative Example 14.

TABLE 7

| | Comp. | | | | Comp. | | | Comp. | |
|---|---|---|---|---|---|---|---|---|---|
| | Ex. 12 | Ex. 10 | Ex. 11 | Ex. 12 | Ex. 13 | Ex. 13 | Ex. 14 | Ex. 14 | Ex. 15 |
| Zinc Chloride | — | 0.0025 | 0.0005 | 0.0001 | — | 0.0025 | 0.0005 | — | 0.0025 |
| Orthoboric Acid²⁾ | 0.5 | 0.5 | 0.5 | 0.5 | 0.5 | 0.5 | 0.5 | 0.5 | 0.5 |
| Borax | 0.02 | 0.02 | 0.02 | 0.02 | 0.02 | 0.02 | 0.02 | 0.02 | 0.02 |
| Sodium Chloride | 0.7 | 0.7 | 0.7 | 0.7 | 0.7 | 0.7 | 0.7 | 0.7 | 0.7 |
| PHMB¹⁾ | 0.0001 | 0.0001 | 0.0001 | 0.0001 | 0.0001 | 0.0001 | 0.0001 | 0.00002 | 0.00002 |
| | (1 ppm) | (1 ppm) | (1 ppm) | (1 ppm) | (1 ppm) | (1 ppm) | (1 ppm) | (0.2 ppm) | (0.2 ppm) |
| HCO60 | 0.05 | 0.05 | 0.05 | 0.05 | — | — | — | — | — |
| Polysorbate 80 | — | — | — | — | 0.05 | 0.05 | 0.05 | 0.05 | 0.05 |
| Hydrochloric Acid | q.s. | q.s. | q.s. | q.s. | q.s. | q.s. | q.s. | q.s. | q.s. |
| Sodium Hydroxide | q.s. | q.s. | q.s. | q.s. | q.s. | q.s. | q.s. | q.s. | q.s. |
| Purified Water | Bal. | Bal. | Bal. | Bal. | Bal. | Bal. | Bal. | Bal. | Bal. |
| pH | 7.0 | 7.0 | 7.0 | 7.0 | 7.0 | 7.0 | 7.0 | 7.0 | 7.0 |

Units of Blending Proportions: w/v %.
¹⁾“PHMB”: Hydrochloride of polyhexamethylene biguanide (compound represented by the general formula (II), wherein n is 12 on average), was used.
²⁾“Orthoboric Acid” is accepted as the standard of “boric acid” by The Japanese Pharmacopeia Sixteenth Edition.

The results are shown in FIG. 7. As is clear from FIG. 7, the ophthalmic compositions containing zinc chloride together with PHMB at each concentration (Examples 10 to 15) had remarkable increase in the TER values, as compared to the ophthalmic compositions containing PHMB alone (Comparative Examples 12 to 14), so that it was clarified that the corneal barrier functions were improved.

### Referential Test Example 2

### Evaluation (4) of Corneal Barrier Functions

The ophthalmic composition was prepared in accordance with the following Table 8, and the influences on the corneal barrier functions were evaluated.

The transepithelial electric resistance value (TER value) was measured in the same manner as in Test Example 8, and the increase ratio was calculated on the basis of the formula (1). Here, the comparative example corresponding to Comparative Example 16 is Comparative Example 15, and the comparative example corresponding to Comparative Example 18 is Comparative Example 17.

TABLE 8

| | Comp. | Comp. | Comp. | Comp. |
|---|---|---|---|---|
| | Ex. 15 | Ex. 16 | Ex. 17 | Ex. 18 |
| Zinc Chloride | — | 0.0025 | — | 0.0025 |
| Orthoboric Acid²⁾ | 0.5 | 0.5 | 0.5 | 0.5 |
| Borax | 0.02 | 0.02 | 0.02 | 0.02 |
| Sodium Chloride | 0.7 | 0.7 | 0.7 | 0.7 |
| PHMB¹⁾ | 0.0001 | 0.0001 | 0.0001 | 0.0001 |
| | (1 ppm) | (1 ppm) | (1 ppm) | (1 ppm) |
| HCO60 | 0.05 | 0.05 | — | — |
| Polysorbate 80 | — | — | 0.05 | 0.05 |
| Hydrochloric Acid | q.s. | q.s. | q.s. | q.s. |
| Sodium Hydroxide | q.s. | q.s. | q.s. | q.s. |
| Purified Water | Bal. | Bal. | Bal. | Bal. |
| pH | 7.0 | 7.0 | 7.0 | 7.0 |

Units of Blending Proportions: w/v %.
¹⁾“PHMB”: Hydrochloride of polyhexamethylene biguanide (compound represented by the general formula (II), wherein n is 12 on average), was used.
²⁾“Orthoboric Acid” is accepted as the standard of “boric acid” by The Japanese Pharmacopeia Sixteenth Edition.

The results are shown in FIG. 8. As is clear from FIG. 8, the ophthalmic composition containing zinc sulfate together with PHMB (Comparative Examples 16 and 18) had rather lowered TER values, as compared to the ophthalmic compositions containing PHMB alone (Comparative Examples 15 and 17), so that it was clarified that the corneal barrier functions were not improved.

### Formulation Examples

The ophthalmic compositions for contact lenses are prepared according a conventional method in accordance with the prescription listed in Table 9.

TABLE 9

| | Formu- | Formu- | Formu- | Formu- | Formu- | Formu- | Formu- | Formu- | Formu- | Formu- |
|---|---|---|---|---|---|---|---|---|---|---|
| | lation | lation | lation | lation | lation | lation | lation | lation | lation | lation |
| | Example 1 | Example 2 | Example 3 | Example 4 | Example 5 | Example 6 | Example 7 | Example 8 | Example 9 | Example 10 |
| Zinc Chloride | 0.0025 | 0.0025 | 0.005 | 0.015 | 0.0025 | 0.001 | 0.001 | 0.005 | 0.005 | 0.01 |
| Polyhexamethylene | 0.0001 | 0.0001 | 0.0001 | 0.0001 | 0.0001 | 0.0001 | 0.0001 | 0.001 | 0.001 | 0.001 |
| Biguanide | | | | | | | | | | |
| EDTA¹⁾ | — | — | — | — | — | 0.05 | — | — | — | — |
| Polyhexanide | 0.0001 | 0.0001 | — | 0.0001 | 0.0001 | — | 0.0001 | 0.0001 | — | 0.0001 |
| Hydrochloride | | | | | | | | | | |
| HCO60²⁾ | 0.2 | 0.2 | 0.05 | — | — | 0.2 | 0.05 | — | — | 0.1 |
| Polysorbate 80 | — | — | — | 0.1 | 0.05 | — | — | 0.1 | 0.05 | — |
| Polyoxyethylene- | 0.05 | — | — | 0.05 | 0.05 | — | 0.05 | 0.05 | — | — |
| Polyoxypropylene | | | | | | | | | | |
| Glycol | | | | | | | | | | |
| Hydroxypropyl | 0.2 | 0.2 | 0.05 | 0.2 | 0.2 | — | — | 0.1 | 0.2 | 0.2 |
| Methyl Cellulose | | | | | | | | | | |
| Orthoboric Acid | 0.5 | 0.5 | 0.5 | 1.0 | 1.5 | 0.5 | 0.5 | 1.0 | 1.8 | 0.5 |
| Borax | 0.02 | 0.02 | 0.02 | 0.1 | 0.2 | 0.02 | 0.02 | 0.2 | 0.4 | 0.02 |
| Hydrochloric Acid | q.s. | q.s. | q.s. | q.s. | q.s. | q.s. | q.s. | q.s. | q.s. | q.s. |
| Sodium Hydroxide | q.s. | q.s. | q.s. | q.s. | q.s. | q.s. | q.s. | q.s. | q.s. | q.s. |
| Purified Water | Bal. | Bal. | Bal. | Bal. | Bal. | Bal. | Bal. | Bal. | Bal. | Bal. |
| pH | 7.0 | 7.0 | 7.0 | 7.5 | 7.0 | 7.0 | 7.0 | 6.5 | 6.0 | 7.0 |

Units of Contents: w/v %.
¹⁾EDTA: disodium ethylenediaminetetraacetate dihydrate
²⁾HCO60: polyoxyethylene hydrogenated castor oil 60

1. An ophthalmic composition characterized in that the ophthalmic composition comprises (A) a biguanide-based disinfectant and (B) zinc chloride.

2. The ophthalmic composition according to claim 1, wherein the biguanide-based disinfectant (A) is a compound having at least one biguanide group represented by the formula: —NH—C(═NH)—NH—C(═NH)—NH—.

3. The ophthalmic composition according to any of claim 1 or 2, further comprising (C) at least one member selected from the group consisting of boric acid and salts thereof.

4. The ophthalmic composition according to claim 3, wherein the component (C) is contained, calculated as a boron atom content, in a proportion of from 0.0001 to 0.045 mol/100 mL.

5. The ophthalmic composition according to claim 1, further comprising a nonionic surfactant.

6. The ophthalmic composition according to claim 1, wherein a total content of the component (B) is from 10 to 50,000 parts by weight, based on 100 parts by weight of a total content of the component (A).

7. The ophthalmic composition according to claim 1, wherein the ophthalmic composition is an ophthalmic composition for contact lenses.

8. The ophthalmic composition according to claim 5, wherein the contact lenses are soft contact lenses.

9. The ophthalmic composition according to claim 1 or 2, for use in disinfection against Acanthamoeba.

10. The ophthalmic composition according to claim 1, wherein the ophthalmic composition is in a pharmaceutical dosage form that is directly applicable to a corneal surface.
